(19) United States
(12) Patent Application Publication
(10) Pub. No.: US 2025/0375146 A1
(43) Pub. Date: Dec. 11, 2025

(54) SYSTEMS AND METHODS FOR CORTICAL STIMULATION AND MAPPING

(71) Applicant: Theranova & The Regents of the University of California, San Francisco, CA (US)

(72) Inventors: Mitchel S. BERGER, San Francisco, CA (US); Emerson Cheung QUAN, Hillsborough, CA (US); Cory SYLLIAASEN, San Francisco, CA (US); Veeshal H. PATEL, San Francisco, CA (US); Alexander VERGARA, San Francisco, CA (US); Anu PARVATIYAR, San Francisco, CA (US); Daniel R. BURNETT, San Francisco, CA (US); John CLARK, San Francisco, CA (US); Lorenzo BUCCI, San Francisco, CA (US); Kondapavulur Tirumala VENKATESWARA-RAO, San Francisco, CA (US); Sohail DESAI, Sacramento, CA (US)

(73) Assignee: Theranova & The Regents of the University of California, San Francisco, CA (US)

(21) Appl. No.: 19/233,518

(22) Filed: Jun. 10, 2025

Related U.S. Application Data
(60) Provisional application No. 63/658,559, filed on Jun. 11, 2024.

(51) Int. Cl. A61B 5/00; A61B 5/395; G16H 40/63

(52) U.S. Cl. CPC A61B 5/4064; A61B 5/395; G16H 40/63

(57) ABSTRACT

Systems and methods for cortical stimulation and mapping are described. In one variation, an apparatus for creating a closed-loop response feedback for an automated brain mapping system may generally comprise a neural stimulator, a console docking station wirelessly communicating with the neural stimulator, and one or more human response sensors configured to interface with the console docking station, wherein said one or more human response sensors are triggered in response to an electrical signal delivered by said neural stimulator.

### CROSS-REFERENCE TO RELATED APPLICATIONS

This application claims the benefit of priority to U.S. Prov. App. 63/658,559 filed Jun. 11, 2024, which is incorporated herein by reference in its entirety.

### INCORPORATION BY REFERENCE

All publications and patent applications and issued patents mentioned in this specification are herein incorporated by reference to the same extent as if each such individual publication, patent application or patents were specifically and individually indicated to be so incorporated by reference.

### FIELD OF THE INVENTION

The present apparatus and methods relate generally to electrical brain mapping.

### BACKGROUND OF THE INVENTION

Brain tumor resection of the eloquent cortices (language, visual, sensory, and motor regions) of the brain pose a number of challenges from a neurosurgical perspective, namely a significant risk of damage to key functional areas of the brain during removal of the tumor. When considering any surgical intervention for a brain tumor, both the surgeon and patient must evaluate the risks and benefits of surgery, and the surgeon must maintain the fundamental tenet of Neurosurgery: to preserve function. However, there is a considerable survival and functional benefit for patients who successfully undergo total resection of the tumor. The challenge of limiting the risk of severe brain injury during resection while performing maximum resection of a tumor can be addressed by cortical stimulation to identify functional areas of the brain during open surgery. Currently, delineating safe margins of resection during open brain surgery involve cortical mapping and intraoperative electrical stimulation of the brain to create neurological impulses which are then closely monitored to inform the surgeon of critical areas. Unfortunately, the tools used to map the brain during surgery have not advanced significantly in the last 15 years, and technological limitations continue to impede optimal resection of brain tumors.

### SUMMARY

Current cortical stimulation instruments require an expensive disposable probe combined with a large generator that can only be controlled from outside of the surgical field. We have combined the instrumentation into a handheld programmable stimulation generator which the surgeon can control in his or her hand. The surgeon can modify the electrical pulse parameters and receive real-time feedback. The disposable tip is attached to this generator and is sterile and comes in contact with the brain. The disclosed technology combines biphasic and monophasic pulses and improved functionality in tailoring the pulse amplitude, frequency, duration, and current to achieve a much more precise stimulation to the brain and feedback to the surgeon, while enabling faster and improved resection with fewer intraoperative seizures. While existing technology can generate a response in a 5 mm-1 cm radius, embodiments disclosed herein are precise enough to give the surgeon precision down to 1 mm during critical portions while permitting faster global mapping during the remainder of the procedure. Some embodiments disclosed herein also allow cortical and subcortical stimulation through a smaller incision and at a lower cost than current devices.

Some embodiments include adjunct functionalities on the cortical stimulator platform to utilize the feedback data to better create real-time mapping images for surgeons to assist decision making and tumor resection without the assistance of a neurophysiologist. Some embodiments include motor-evoked potential monitoring systems (to connect the brain stimulation to external monitoring). Some embodiments include the automated stimulation and detection of evoked potentials or evoked responses using a hand-held stimulator with high- and low-frequency waveforms for cortical and subcortical mapping, once the surgeon makes contact with brain tissue. Some embodiments include artificial intelligence and/or machine learning (AI/ML) algorithms to detect evoked functional response. Some embodiments include a suite of monitoring systems to add an additional layer of validity to the electrophysiologist in the operating room. Some embodiments may be used for the treatment of epilepsy, other brain or mental or neurological disorders or diseases

### BRIEF DESCRIPTION OF THE DRAWINGS

The drawings constitute a part of this specification and include exemplary embodiments of the invention, which may be embodied in various forms. It is to be understood that in some instances various aspects of the embodiments may be shown exaggerated or enlarged to facilitate an understanding of the embodiments.

FIG. 1 shows an embodiment of the stimulation device handset.

FIG. 2 shows an embodiment of the hand-held stimulation device with a sterile sheath.

FIG. 3 shows an embodiment of the hand-held stimulation device in use stimulating the tissue of the brain.

FIGS. 4A and 4B show possible embodiments of a user interface on the device.

FIG. 5 shows an embodiment of the docking station device which includes EMG sensing.

FIG. 6 shows an embodiment of the hand-held stimulation device which is used for monopolar stimulation.

FIG. 7 shows the device in use on a patient along with EMG electrodes on the patient's extremities and the docking station, where the electrode hub is connected to the docking station.

FIG. 8 shows an embodiment of the device where the connection between the docking station and the electrode hub is wireless.

FIG. 9 shows an embodiment of the device where monopolar stimulation is being used for brain mapping.

FIG. 10 shows an example of a display/docking station.

FIG. 11 shows an example of evoked responses to a set of stimuli applied to the patient's brain.

FIG. 12 shows an overlay of the 89 responses captured by the “Left Abductor Pollicis Brevis” sensor using different stimulus intensities applied in different locations of the brain.

FIGS. 13-15 show evoked responses of increasing magnitude captured on the left abductor pollicis brevis sensor.

FIG. 16 shows distinct power spectrum for a: negative response from FIG. 13 (blue), low positive response from FIG. 14 (orange) and high positive response from FIG. 15 (green) (limited between 0 and 1 [KHz]).

FIG. 17 shows a schematic representation of the overall system, showing the interconnections between the components and the patient, for the automated algorithms used to detect functional thresholds after stimulation of brain tissue.

FIG. 18 shows the logic details for algorithm 1 in FIG. 17.

FIG. 19 is a block diagram of a data processing system, which may be used with any embodiment of the invention.

FIG. 20A and B show examples of the stimulation system used in mapping the image processing, language and cognitive processing centers of the brain.

FIG. 21 shows the workflow for mapping the image processing, language processing and cognitive centers of the brain.

FIGS. 22A and 22B show the timing sequences for implementation during mapping of the image processing, language processing and cognitive centers of the brain.

### DETAILED DESCRIPTION

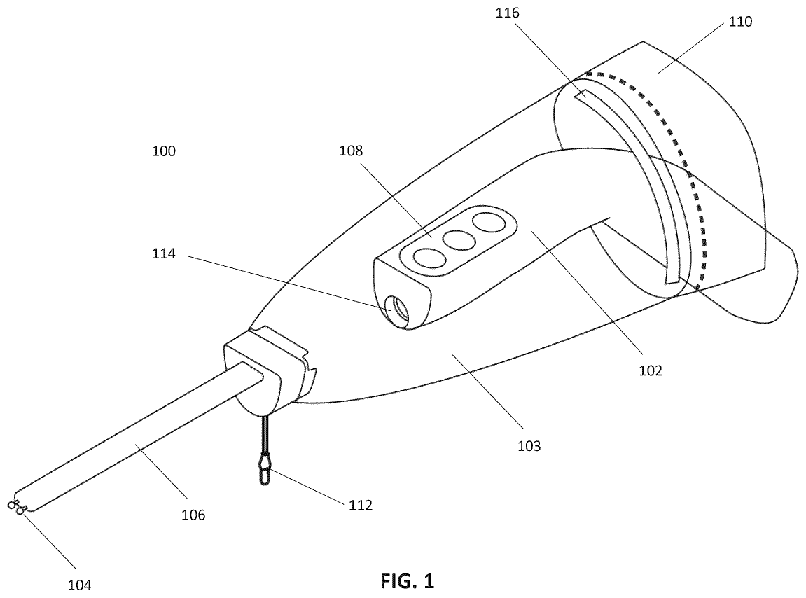

FIG. 1 shows an embodiment of stimulation/mapping device handset or handpiece 100. It may be used in conjunction with a docking station for brain mapping during surgery.

The stimulation/mapping system may include a wireless (or wired), handheld waveform generator, a docking station with touchscreen tablet and/or other display/control, a sterile disposable electrode probe tip, a sterile sheath, and a sterile ground wire. The handheld device may contain functional electronics, battery/power supply, user-specified controls, and a disposable probe tip. The handheld stimulator may be wirelessly connected to the docking station allowing a neurosurgeon to control electrical stimulation intensity during mapping of cortical and subcortical regions of the brain to map the functional pathways (identify areas of motor, sensation and speech functionality) of the brain prior to tumor resection during open brain surgery. The surgeon can adjust the amplitude of stimulation on the handheld device and deliver stimuli with a press of a button. A neurophysiologist or neurosurgeon can switch stimulation protocols to the handheld device through a companion tablet/docking station that may communicate wirelessly via Bluetooth or other wireless technologies.

The stimulator/mapping handpiece or handset embodiment shown in FIG. 1 includes casing 102, stimulating electrodes 104, pliable, or controllable tip 106, monopolar return electrode 112, control buttons 108, stimulator connector 114, sterile sheath seal 116, and sheath detachable portion 110.

The casing may be made out of any suitable material including polymer and may not need to be sterilized. Tip 106 may be steerable, flexible, rigid etc., and may be detachable from the casing. The tip may be sterilizable and/or disposable. In use, a sterile disposable tip may be attached to the casing. The casing may be encapsulated within a sterile sheath so that the device in use is sterile. When enclosing the casing in the sterile sheath, it may be useful to have a detachable portion which can be touched by a non-sterile hand, and then removed by the non-sterile hand before sealing the sheath via the sterile sheath seal. This allows a non-sterile person to help place the sterile sheath on the device.

The tip may include one, two, or more electrodes which deliver current to tissue, such as brain tissue. The tip may also include one or more sensors, and/or other functions. The control buttons may be used to initiate stimulation between two electrodes on the tip when the electrodes are in contact with tissue, such as brain tissue. The device may be used in either biphasic mode or monophasic mode. In biphasic mode, the polarity of the current is reversed each half-cycle through the stimulation pulse period. In monophasic mode, the polarity is not reversed. The stimulator may be used by either left-handed or right-handed users.

The control/display area of the device may include a touch screen, buttons, displays etc. settings may include stimulation current amplitude, mode (i.e. monophasic or biphasic), pulse frequency, pulse duration, tip angle, display settings, etc. These parameters may be controlled by the user and/or displayed.

Stimulator connector 114 allows the sterile tip to come into electrical connection with the rest of the handset. In some embodiments, this connection happens through a sterile sheath, while maintaining the sterility of tip 106, as well as casing 102, which is enveloped by sterile sheath 103. The sheath may be held in place via sterile sheath seal 116. The handset may be wireless or wired, and controlled by a controller, which in some embodiments, may be the docking station, which may be part of the handset, or remote. In embodiments where the handset is wired, the sterile sheath may cover all or part of the wires leading back to an electrical ground, power supply or generator. In embodiments where the handset is wireless, the handset may be powered by batteries.

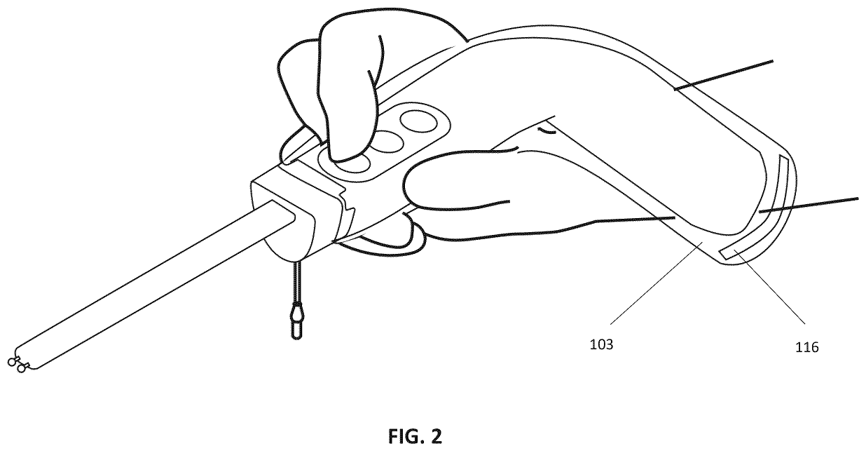

FIG. 2 shows a similar embodiment of the hand-held stimulation device with a sterile sheath over it to enable use during surgery. The device is wirelessly connected to the docking station and battery operated. Sterile sheath seal 116 may alternatively be a band, clip, seal, etc.

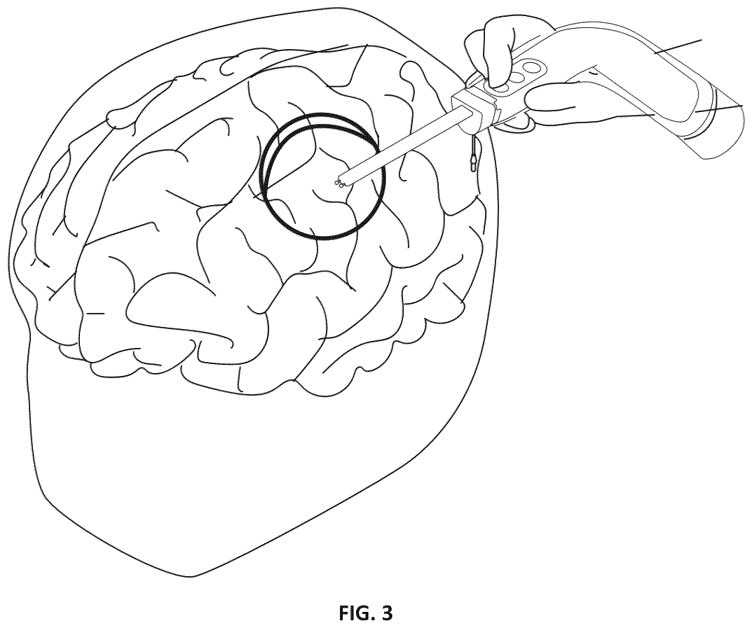

FIG. 3 shows an embodiment of the stimulation device in use stimulating the tissue of the brain.

Some embodiments of the stimulation device may include a reusable battery. Some embodiments may include the ability to charge the battery via inductive charging.

Some embodiments of the stimulation device may include a sensing function. For example, the device may include the ability to measure tissue impedance. This sensing may be performed with the same electrodes that apply stimulation or via separate electrodes or sensors to measure the evoked motor, speech, visual, smell, sense and other functional response. Other sensors may include temperature, electromyography (EMG), electroencephalography (EEG), electrocorticography (ECoG), chemical, pressure, force, motion, etc. These sensed parameters may be used to evaluate the health of the patient, the health of the tissue, differentiate between healthy and tumorous tissue, assess whether the stimulation is being adequately delivered to the tissue, etc.

For example, impedance measurements may be used to help identify variability in the resistance of the tissue in contact with the stimulator—this may enable the determination of any correlation between healthy versus cancerous tissue.

Alternatively, impedance measurements may be used to determine the force of tissue contact necessary for adequate stimulation.

In some embodiments, the current delivered to the tissue is measured via an ammeter or other methods.

In some embodiments, the stimulation button is pressed before the stimulating electrodes contact the brain tissue. In some embodiments, the stimulation button is pressed after the stimulating electrodes contact the brain tissue.

In some embodiments, the handset is in wireless, i.e. Bluetooth, communication with a handheld computer/controller, such as a tablet or mobile phone. The computer may alternatively be non-handheld or remote. The computer may act as a controller and/or a display. The computer may allow modifying of settings etc.

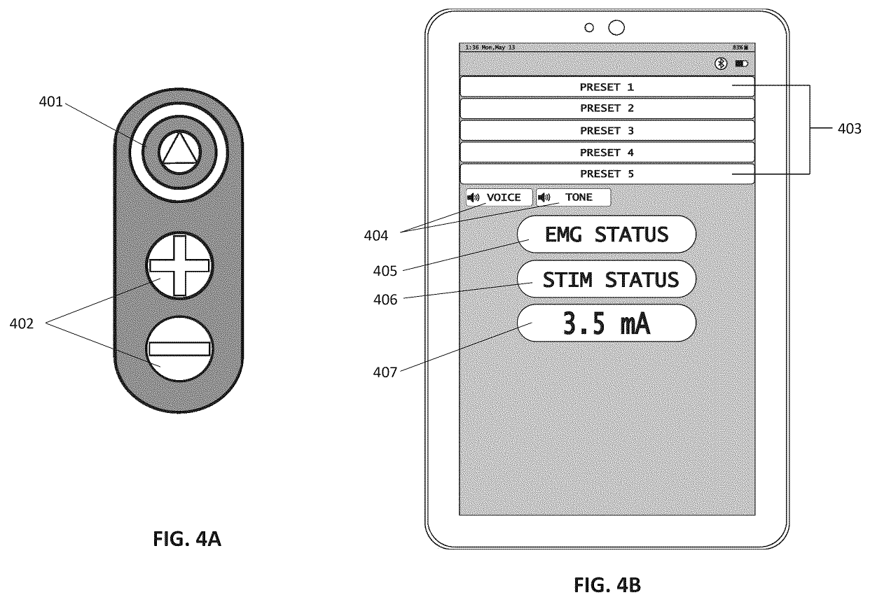

FIGS. 4A and 4B show possible embodiments of a user interface on the device and accompanying tablet. FIG. 4A shows some example areas including a trigger button 401. The trigger button may be used to initiate delivery of stimulus current to the target tissue. In some embodiments, the trigger button may also be used to select a preset stimulation profile with preset parameters for frequency, pulse width, pulse count, mode of operation (bipolar/monopolar), etc. Stimulus current amplitude setting may be controlled by plus and minus buttons 402.

FIG. 4B shows a possible embodiment of a user interface on a tablet or docking station. In this embodiment, output waveform parameters may be selected via preset buttons 403. Presets may include settings such as pulse frequency, pulse width, pulse polarity, mode of operation (bipolar/monopolar), pulse count. The user interface may also include buttons 404 to control audio feedback such as voice and tones. The user interface may also include indications on the status of sensors such as EMG status indicator 405, the handheld stimulator status indicator 406, and the output current amplitude indicator 407.

Some embodiments include a visual alert near the tip of the device, such as lights which may indicate different measured parameters. For example, a green light may indicate that the delivered current is within a threshold of an acceptable percentage of the set current level. A red light may indicate that the delivered current is outside of a threshold of an acceptable percentage of the set current level. An orange light may indicate that the delivered current is approaching the outside of a threshold of an acceptable percentage of the set current level. Lights may also indicate temperature, hydration level, impedance or any level of any other measurable parameter. Lights may also indicate battery level of the device. Lights may also indicate whether the device is malfunctioning in any way. Alternatively, an auditory, or vibration alert may alert any of the parameters or the battery level or functional status.

Some embodiments may include some of the following features:
Effectively deliver bipolar and monopolar stimulation from 0.5 mA-20 mA at an interval of 0.5 mA
Handheld form factor and wireless operation
Battery life of 12 hours in continuous use
Device can be fully charged overnight.
Can be fully charged via a docking station
Can deliver stimulation at 60 Hz frequency
Can deliver stimulation at 50 Hz or 60 Hz, and can be easily toggled by the surgeon during the procedure.
Can deliver stimulation frequency of 1 Hz to 1 KHz with a 1 Hz resolution.
Can deliver stimulation at a pulse width of 0.5 ms to 2 ms.
Can maintain sterility throughout the procedure and be cleaned between procedures.
Incorporates a disposable component that can be easily and quickly integrated with sterile sheath to establish and maintain a sterile barrier throughout the procedure.
Includes visual or auditory indication of successful current delivery and/or battery status.
Includes visual or auditory indicator of successful current delivery, partial current delivery, device malfunction, contact with tissue, and/or battery/charge status.
Has a display mechanism to show current setting and current delivered.
Ergonomic handheld form factor and weight less than 2.2 lbs.
Has a display mechanism to toggle between various setting parameters, including current amplitude, pulse duration, and pulse frequency, as well as current delivered.
Has wireless connectivity to tablet or external device to record stimulation parameters, delivery, and feedback.
Integrates with other devices used in surgery such as an Electronic Medical Record (EMR) systems or other EMG or intra-operative neuromonitoring systems.

In some embodiments, the electrodes at the tip of the device spread into a Y-shape, so that they may be more easily seen during use.

In some embodiments, the distance between electrodes can be adjusted manually, or with a special tool. Alternatively, tips may be available with different electrode distances, sizes and/or configurations.

Some embodiments include the ability to mark the tissue. For example, a biocompatible dye may be applied by the device to the tissue to mark tumor vs. healthy tissue, or tissue to avoid, etc. The dye may be colored and the device may have the capability of applying different color dyes to mark different areas.

Some embodiments include the ability to integrate with other visualization systems for virtual marking.

Some embodiments include the ability to integrate with magnetic resonance imaging (MRI) imaging equipment, navigation equipment, robotic guidance and surgical equipment or other imaging equipment, for spatial orientation.

Some embodiments of the device system may include the ability to obtain physical patient response due to the stimulation. For example, accelerometers or other sensors may be used on hands, feet, face or other body location to sense twitches or tremors or seizures due to stimulation. The system may include the ability to collect feedback due to eyesight changes, movement, taste, feel, hearing, speech, language, verbal and/or smell. This feedback may be collected from the user of the device or directly from the patient. The feedback may be collected from a touch interface, voice interface, visual interface etc.

Some embodiments of the device include the ability to stop a tremor or seizure caused by stimulating the brain tissue by applying a cooling medium to the surface of the brain, such as cold saline, Ringer's lactate, etc. This application may be initiated manually, or automatically via the sensing of a tremor or seizure. The application may be localized, as with a squirting mechanism, or more general, as with a bathing mechanism. The cold fluid may exit the device via the tip. The cold fluid reservoir may be in the tip of the device, or in the handset of the device, or located elsewhere. For example, the fluid reservoir may be incorporated into the sterile sheath. Some embodiments may include the ability to apply a counter stimulation to stop a tremor or seizure caused by stimulating the brain tissue.

Some embodiments of the device may include monopolar stimulation or bipolar stimulation.

Some embodiments of the device may include monopolar stimulation and bipolar stimulation. For example, the bipolar stimulation may have a relatively low frequency range, and the monopolar stimulation may have a relatively high frequency range. For example, the device may have monopolar stimulation capabilities of around 250 Hz (0.004 second period), with a train of 5 pulses (1-2 trains/second). In another example, the device may have monopolar stimulation capabilities of around 200-300 Hz. In another example, the device may have monopolar stimulation capabilities of around 150-250 Hz. In another example, the device may have monopolar stimulation capabilities of around 250-350 Hz. The bipolar stimulation capabilities may be around 50 Hz to 60 Hz. Alternatively, the bipolar stimulation capabilities may be around 40 Hz to 60 Hz. Alternatively, the bipolar stimulation capabilities may be around 50 Hz to 70 Hz. Alternatively, the bipolar stimulation capabilities may be around 20 Hz to 80 Hz.

In embodiments where monopolar stimulation is an option, the stimulating electrode is at the tip of the device, and the return electrode is somewhere in communication with the patient's body. The lead from the return electrode may be connected to the handset.

Some embodiments of the device may include EMG (or EEG) sensing, for example, for sensing compound muscle action potentials (CMAPs) in response to cranial stimulation. By sensing CMAPs, the EMG sensors can detect motor function as a result of stimulating different areas of the brain. The EMG electrodes may include electrodes that are placed anywhere on the body. The EMG electrodes may be integrated with the device. In some embodiments, the feedback from the EMG electrodes may control the stimulation parameters (see FIG. 17). In some embodiments, the stimulation parameters may be adjustable remotely, by a user other than the physician performing the cranial stimulation, for example via the display (see FIG. 7 and other FIGS.).

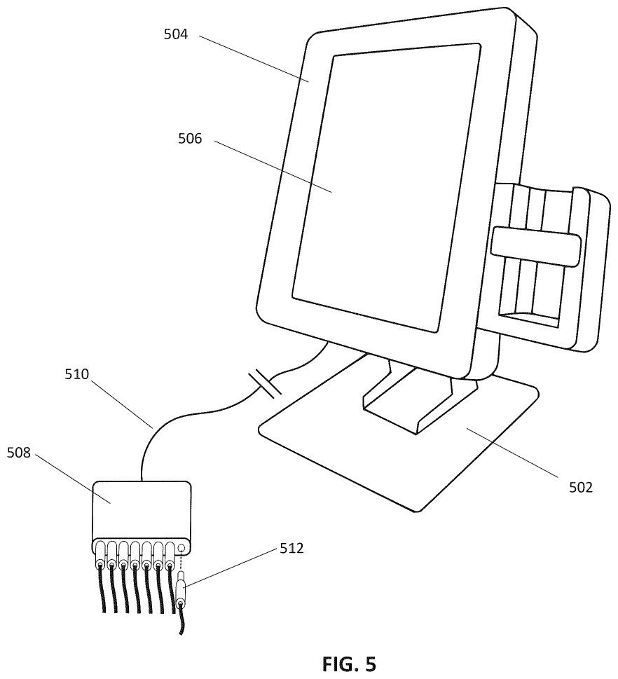

FIG. 5 shows an embodiment of the docking station which includes EMG sensing. Shown here are docking station stand 502, docking station case 504, docking station touch screen display 506. Also shown is electrode connector hub 508.

Electrode hub 508 may connect EMG electrodes to the docking station. This connection may be wireless or wired, as shown here, via wire 510. In wireless connections, electrode connector may include wireless transmission, and/or reception capabilities, such as Bluetooth, fiber optic, infra-red (IR) or other capabilities. EMG electrode leads may connect to the electrode hub via adapters 512. The docking station may include electronics, including controller electronics, such as data acquisition and/or analog front-end circuitry. These electronics may include signal conditioning circuitry, signal amplification circuitry, signal filtering circuitry, signal converting circuitry (i.e. analog to digital conversion) etc.

In some embodiments Electrocorticography (ECOG) electrodes or scalp electrodes may be placed on the patient to perform signal averaging in order to monitor evoked potentials of neural origin, such as cortico-cortical evoked potentials.

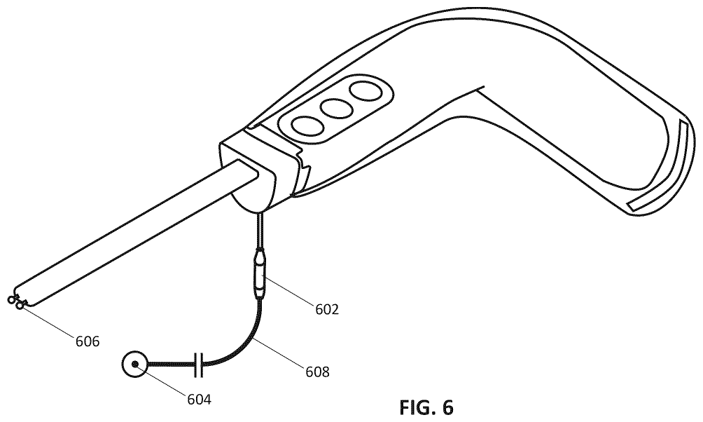

FIG. 6 shows an embodiment of the hand-held stimulation device which is used for monopolar stimulation. In this embodiment, electrode connector 602, connects ground electrode 604 to the device via wired monopolar lead connection 608. In this configuration, the device can be used for bipolar stimulation and/or monopolar stimulation. During monopolar stimulation, one of electrodes 606 may be rendered inactive, while the other electrode is used for the monopolar stimulation, with electrode 604 used as the ground electrode. The two electrodes 606 may be color coded, or otherwise identified so that the user knows which electrode is active during monopolar stimulation. The user may have the ability to toggle between monopolar stimulation and bipolar stimulation during the procedure. This toggling is preferably able to be done without moving the device, for example, with a button on the docking station, by audible command, foot pedal, etc.

Ground electrode 604 may be a patch electrode, a needle electrode or other type of electrode. The ground electrode may be placed on the patient's forehead, or elsewhere on the patient.

FIG. 7 shows the device in use on a patient along with EMG electrodes on the patient's extremities. EMG electrodes 702 are connected to electrode hub 710 via electrode leads 706. The hub connects to the docking station 704 via a high data rate wired connection 712 such as USB or other such high-speed digital interconnect. Also shown here is display 708 which may display, or otherwise communicate to the user/physician, the EMG signal responses to the cranial stimulation of the device. For example, the display may be a tablet, or other computer screen.

Display 708 may show representation of body parts, for example, a foot, calf, thigh, hand, arm, biceps, extensor carpi radialis, flexor carpi ulnaris, first, dorsal interosseous, abductor pollicis brevis, abductor digiti minimi, rectus femoris, tibialis anterior and abductor hallucis, etc. of the patient. The display may show a visible display when one or more body parts moves, or exhibits an EMG signal or an evoked potential for other functions, such as speech, vision, etc. The display may only show an indication of a response when the EMG signal or evoked potential is above a certain amplitude.

In some embodiments, accelerometers may be used instead of, or in addition to, EMG electrodes, and movement of the various body parts may be sensed by the accelerometers, and the display communicates these movements to the physician.

The communication of EMG or other signals from the display to the user/physician may be visual, audible, tactile, etc. For example, the display may communicate “right foot” if an EMG signal relating to the right foot is received by the display. The display may further communicate the level of the signal, for example, “right foot minor” or “right foot major”, or similar.

In some embodiments, the display may be in the physician's field of vision, for example, on a wall on the other side of the patient. In some embodiments, the display may be within the microscope view of the physician so that the physician does not need to look up or move his/her head to see the EMG signals or evoked potentials.

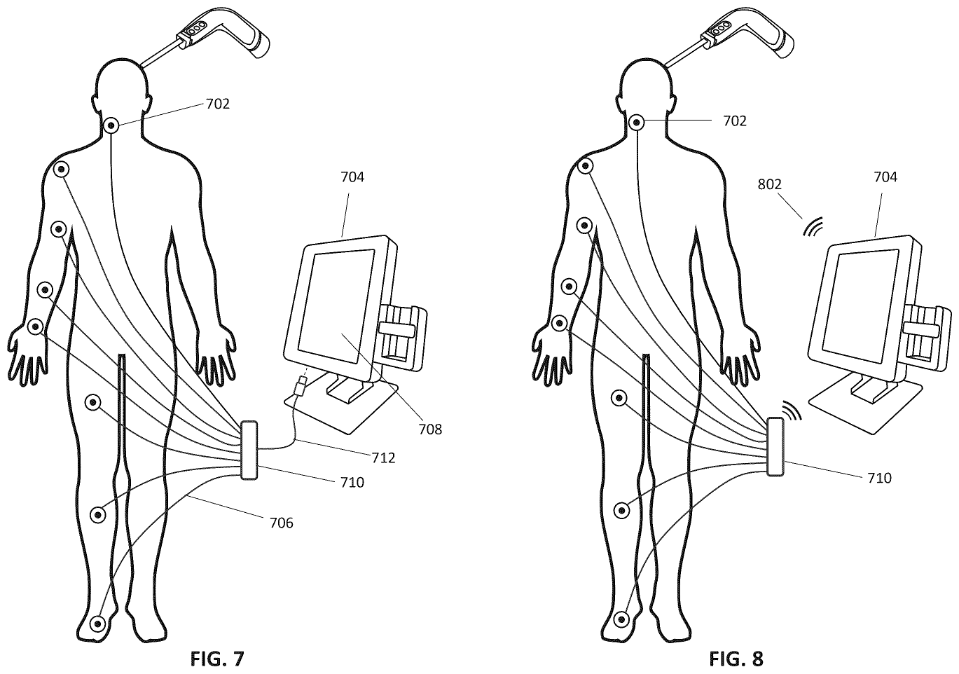

FIG. 8 shows an embodiment of the device where the electrode hub 710. connected to the EMG electrodes 702, communicates with the docking station 704 via a wireless interface, represented by wireless connection 802. The electrode hub wirelessly transmits EMG measurements to the docking station in this embodiment.

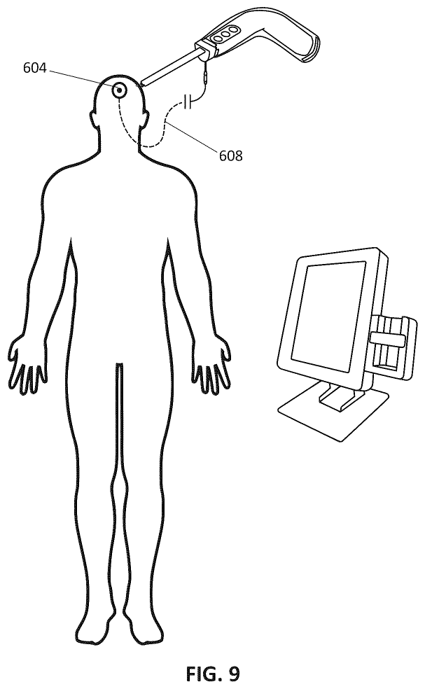

FIG. 9 shows an embodiment of the device where monopolar stimulation is being used.

In some embodiments, the display includes the ability to display and/or control the settings and/or parameters of the device. For example, the display may display, and/or allow for control of, the current setting, the delivered current (current output), the impedance measurements, the force of tissue contact, tissue temperature, device temperature, device battery status, sensed EMG, sensed EEG, sensed ECoG, sensed chemical attributes, force or pressure of the device against the tissue, motion of the device with respect to the tissue, etc.

The display may display and/or communicate any of these parameters and/or settings, including stimulation parameters etc., via graphical, symbolic, numerical, audible, vibration and/or any other communication methods.

In some embodiments, the display is also a docking station for the device. The display may include the ability to recharge the handpiece, or download/upload data from/to the handpiece.

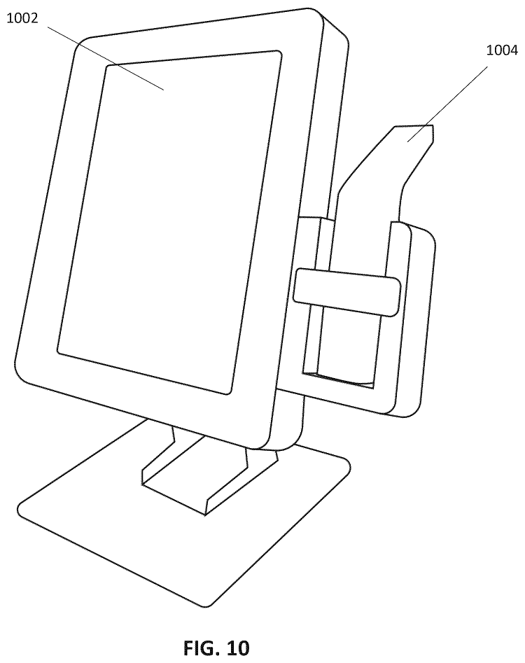

FIG. 10 shows an example of a display/docking station. Shown here is display 1002, which may pivot, may be detachable and/or may be a tablet. Also shown is stimulating handpiece 1004 docked in the display/docking station. Controls may additionally or alternatively be on a touch screen display and/or on the handpiece.

Handpiece settings, including stimulation settings, may be controlled by the display/docking station and/or the handpiece. These controls may be wirelessly communicated to the handpiece.

The display/docking station may also be in communication (wired or wireless) with other devices, such as EMG instrumentation, ECG instrumentation etc. The display/docking station may communicate to and/or receive information from these devices, including the ability to control these devices. For example, the display/docking station may trigger the initiation of EMG recording. These communications may be manual, or automatic, for example triggered by the initiation of stimulation.

The display/docking station may include a touchscreen, which may be in the form of a tablet. The display/docking station may serve as a charging base and/or communication base for the device handset. The display/docking station may allow for remote control and/or display of various stimulation parameters, including pulse intensity, mode (i.e. monophasic or biphasic), pulse amplitude, pulse frequency, pulse duration, tip angle, display settings, as well as EMG or EEG response(s), etc. In some embodiments, parameters may be saved into stimulation protocols which can be selected for later use. More than one stimulation protocol may be stored and the user may switch between protocols before and/or during use.

The display/docking station may allow the user to select a range of stimulation patterns, including low frequency (LF) stimulation, high-frequency (HF) stimulation, monophasic, biphasic, pulse polarity, pulse frequency, pulse width, number of pulses and their combinations for brain mapping. Any combination of these parameters may be combined into a protocol. Alternatively, these stimulation patterns or protocols may be selected on the handpiece. Some embodiments may include the ability to store at least 2 stimulation protocols. Some embodiments may include the ability to store at least 3 stimulation protocols. Some embodiments may include the ability to store at least 4 stimulation protocols. Some embodiments may include the ability to store at least 5 stimulation protocols. Some embodiments may include the ability to store at least 6 stimulation protocols. Some embodiments may include the ability to store at least 7 stimulation protocols. The surgeon can customize programs on the docking station through a broad range of stimulus parameters and adapt it to modern neurophysiology needs by creating adaptable/customizable mapping protocols.

Some embodiments of the device include machine learning capabilities such as threshold searching, waveform analysis and/or other algorithms analysis for identification of evoked functional response and/or seizures. The device may provide instantaneous feedback to the surgeon during the surgical procedure, without need for interactions with the team of specialists. In other words, the neurosurgeon may be in full control during the surgical procedure, i.e., stimulate, receive feedback, resect the tumor continuously without reliance on the support team for mapping. The neurosurgeon may be able to operate alone without dependence on neurophysiologists or other specialists.

Some embodiments disclosed herein also allow cortical stimulation through a smaller incision and at a lower cost than current devices. For example, the procedure may be performed with an incision with a radius of 1 mm or smaller. Alternatively, the procedure may be performed with an incision with a radius of 2 mm or smaller. Alternatively, the procedure may be performed with an incision with a radius of 3 mm or smaller. Alternatively, the procedure may be performed with an incision with a radius of 4 mm or smaller. Alternatively, the procedure may be performed with an incision with a radius of 5 mm or smaller.

Some embodiments allow for faster procedural time with automated stimulation and threshold detection algorithms. Some embodiments include modern waveform analysis with evoked response and/or seizure identification.

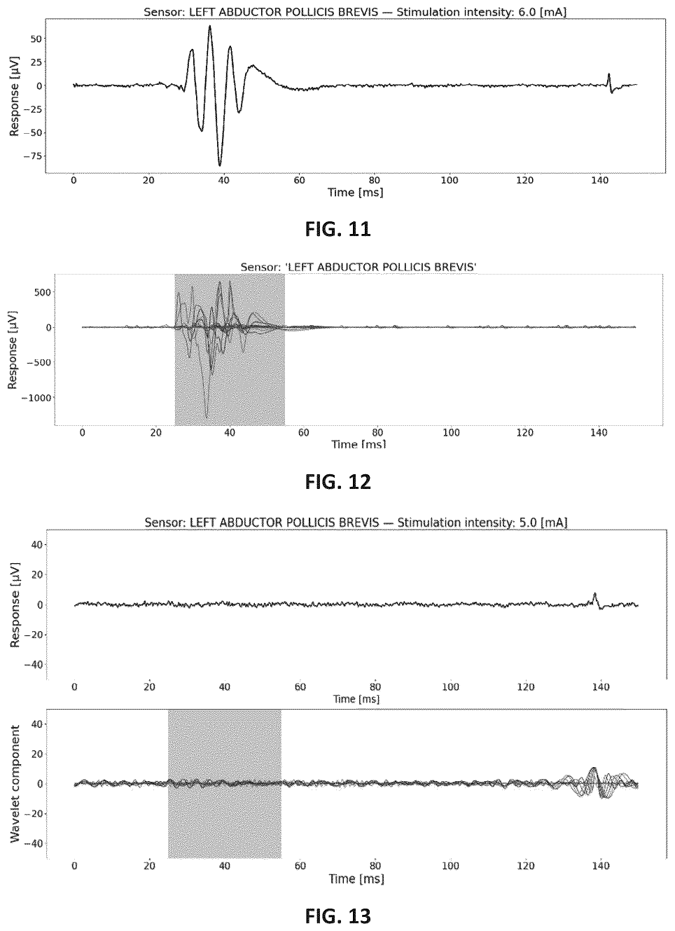

An example of evoked responses to a set of stimuli applied to the patient's brain is shown in FIG. 11. The evoked response shown here has a time duration of 150 [ms] with a sampling period of 120 [μs].

The stimuli that generate the responses represented by FIG. 11 consist of sequences of 5 electrical impulses with a width of 500 [μs] and a period of 5 [ms]. Furthermore, the dataset includes responses generated using stimuli of different intensities, ranging from 0 to 20 [mA]. For each intensity level, one or more stimulations were performed (in different locations of the brain) for a total of 89 stimulations in the dataset. Each stimulation produced 16 different response signals (one for each sensor), for a total of 1424 data samples.

Some embodiments are able to automatically classify evoked responses into two classes: positive responses and negative responses. A positive response is generated by applying an appropriate stimulus to specific locations in the brain that are neurologically related to the muscle where the sensor is applied. Negative responses are instead generated by applying stimuli to tumor areas or areas of the brain that are unrelated to the muscles of interest. Evoked EMG responses recorded from a patient during brain surgery may be classified using wavelet decomposition and/or other methods.

With a stimulus applied at time t=0, we expect its response to be delayed by a certain amount of time which is proportional to the neural distance between the stimulated area in the brain and the position of the sensor on the patient's body. Since the delay between stimulation and its response is known for any sensor position, attention can be focused on a time window of fixed size within which a positive or negative response can be assessed. To understand how such an observation window can be determined for any given sensor, FIG. 12 shows an overlay of the 89 evoked responses captured by the sensor “Left Abductor Pollicis Brevis” from the dataset. It is possible to notice from FIG. 17 that the actual sensor activations are concentrated in the interval between t=25 [ms] and t=55 [ms] which constitutes our observation window for this sensor.

FIGS. 13-15 show evoked responses of increasing magnitude captured on the left abductor pollicis brevis sensor. For each figure, the upper graph shows the actual evoked response, while the lower graph shows its wavelet decomposition using a Gaussian kernel at 8 different scales.

FIG. 13 shows a negative response for the sensor “Left Abductor Pollicis Brevis” (top). The wavelet decomposition of the response using a Gaussian kernel at 8 different scales (bottom).

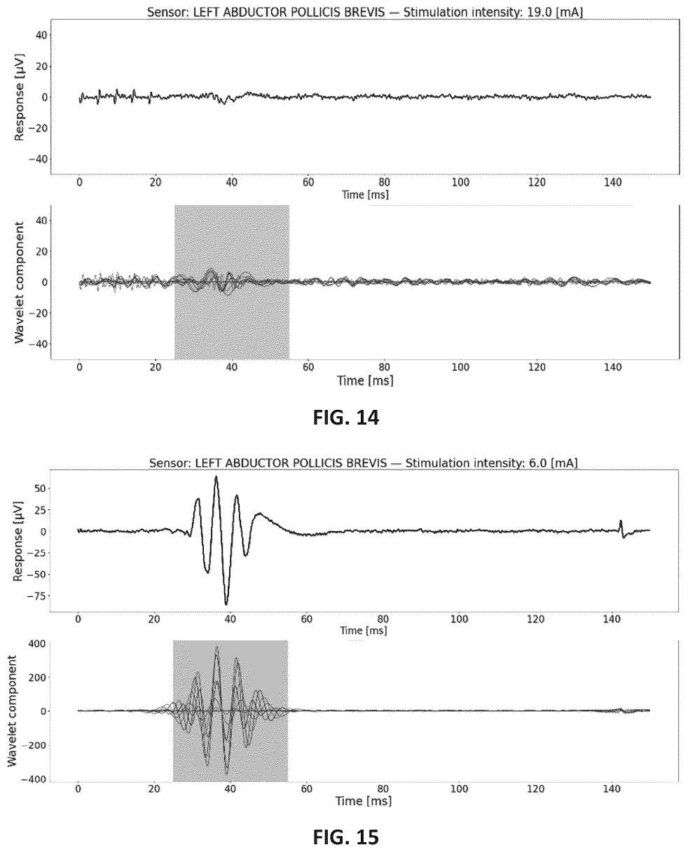

FIG. 14 shows a low entity (positive) response for the sensor “Left Abductor Pollicis Brevis” (top). The wavelet decomposition of the response using a Gaussian kernel at 8 different scales (bottom).

FIG. 15 shows a high entity (positive) response for the sensor “Left Abductor Pollicis Brevis” (top). The wavelet decomposition of the response using a Gaussian kernel at 8 different scales (bottom).

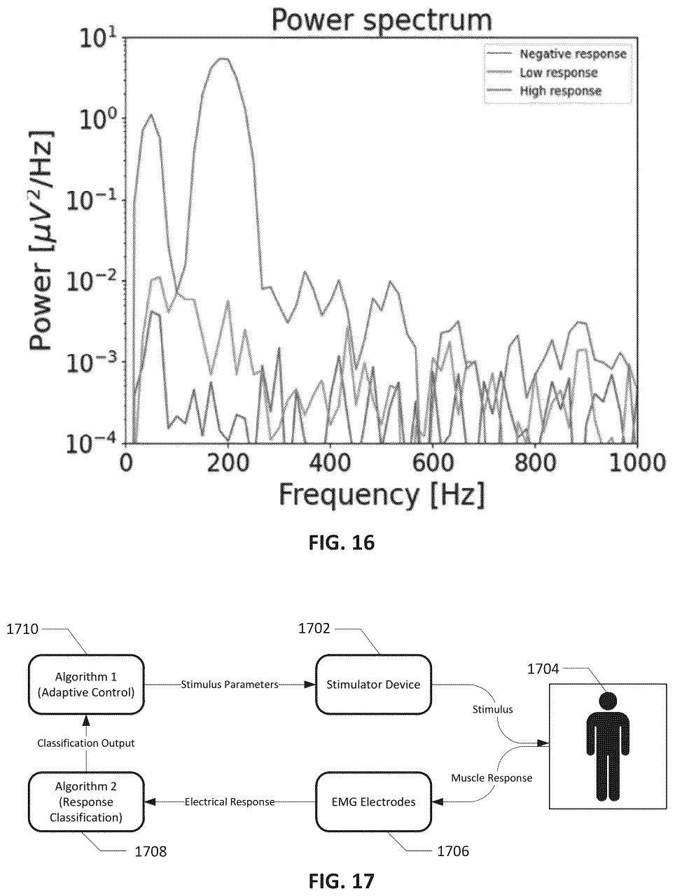

The wavelet decomposition enables location in time of the appearance of specific harmonic frequencies in the evoked responses. By analyzing the wavelet decompositions in combination with the power spectrum, as shown in FIG. 16, it is possible to observe that specific harmonic frequencies (e.g., 60 and 200 [Hz] components) increase in power only within the observation window. Such an increase in power ranges from a factor of ˜2.5 for low-magnitude responses to over ˜600 for high-magnitude responses, with respect to negative responses (in terms of area under the power spectrum curve).

FIG. 16 shows the power spectrum of the: negative response from FIG. 13 (blue), low positive response from FIG. 14 (orange) and high positive response from FIG. 15 (green) (limited between 0 and 1 [KHz]).

Evoked EMG responses and evoked potential and/or response recorded from a patient during brain surgery can be classified on the basis of time and frequency-domain features, which allows for automating threshold search using machine learning and artificial intelligence in some embodiments of the surgeon-directed brain mapping/stimulation system.

FIG. 17 shows a schematic representation of the overall system, showing the interconnections between the components and the subject for automated stimulation and detection of motor thresholds during cortical and subcortical mapping. Shown here is stimulator device handpiece 1702, which stimulates patient 1704, EMG electrodes 1706 which measure the muscle (or evoked potential) response of the patient, algorithm 2 (response classification) 1708, which then feeds algorithm 1 (adaptive control) 1710, which then informs the stimulus parameters of the stimulator device.

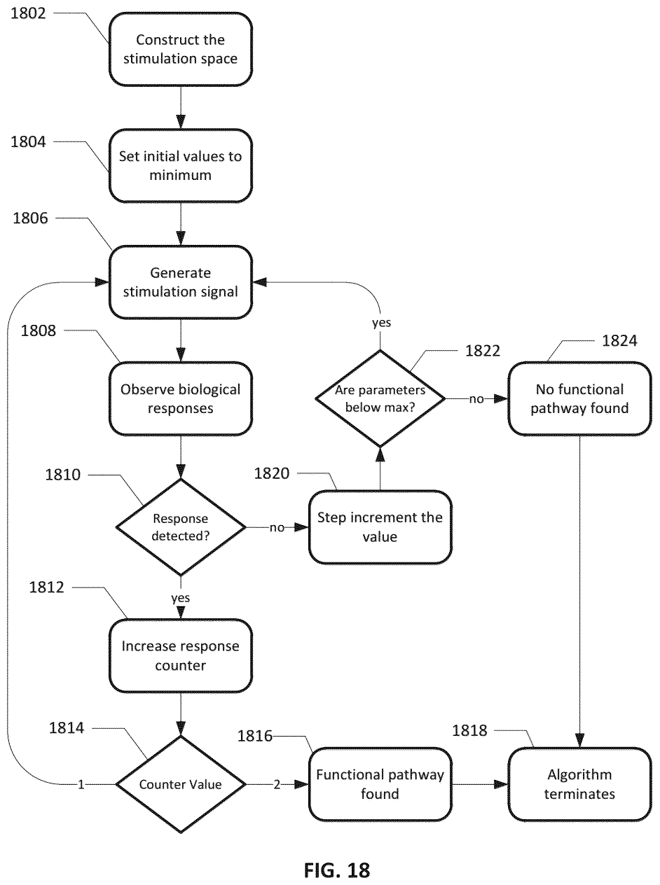

FIG. 18 shows the logic details for algorithm 1 in FIG. 17. Algorithm 1: Adaptive Control for Stimulation Generation: This algorithm consists of two parts. In the first part, the model constructs the stimulation parameter space. In the second part, the model traverses the parameter space to identify the optimal parameters of the stimulation that result in a safe stimulation and evoke a functional response in the neural pathway.

Parameter space estimation: The range of parameters for the stimuli are determined based on the user's (neurosurgeon's and/or neurophysiologist's) input. User's input includes, the number, width, repetition rate, and maximum amplitude of the stimulation pulses used to evoke a functional (motor and other evoked potential) response.

Grid search: A simple linear grid search is performed to identify the best set of parameters characterizing the stimuli. The “best” parameters are those generating a deflection in the waveform (positive evoked motor) response using the minimum stimulus intensity. The goal is to minimize the time required to identify the proper threshold value (i.e., the minimum intensity for the stimulus to generate a perceivable positive response).

The algorithm consists of an iterative criterion where, at each step, one set of parameters is selected from the parameter space, and the corresponding stimulus is generated and applied to the stimulation region. After applying the stimulus, the algorithm waits for a predefined amount of time to receive a detection signal from the machine learning-based response detection unit. This wait time is determined on the basis of some patient's physiological attributes, such as height, weight, etc. When a response is not detected, the algorithm takes the next step in the parameter space and generates the next stimulus. This iterative process repeats (for each given stimulation area) until either a positive response is detected, or the parameter space is exhausted.

FIG. 18 shows the following steps: Constructing the simulation space for a given parameter 1802, setting the initial value to a minimum value 1804, generating the stimulation signal 1806, observing, and/or sensing, the biological response to the stimulation signal 1806, determining whether a biological response is detected 1810. If a response is detected at step 1810, a counter is increased at step 1812, if the counter value is 1, the algorithm repeats the signal generation at step 1806. If the counter value is 2, step 1814 points to step 1816, indicating that a functional pathway has been found, and then terminates the algorithm at step 1818. If a response is not detected at step 1810. If a response is not detected at step 1810, the stimulation parameter value is increased at step 1820. If the stimulation parameter value is below the maximum value at step 1822, the algorithm repeats the signal generation at step 1806 with the new stimulation value. If the stimulation parameter value is at or above the maximum value at step 1822, the algorithm determines that no functional pathway was found at step 1824, and terminates the algorithm at step 1818.

The evoked response detection algorithm (ERDA) described here is an innovative algorithm to detect (subclinical) evoked potentials during cortical and subcortical mapping, which may or may not be detected by muscle movement during EMG. The primary advantage for ERDA is the automation of stimulation and threshold detection (changes in base amplitude), especially in rural and community hospitals (where neurophysiologists or NPs are not available), and in circumstances where the determination is difficult even for NPs, such as when the signal-to-noise ratio (SNR) is poor. While signal averaging is standard practice to optimize SNR, ERDAs can significantly extend the time to acquire a usable signal (noise reduction is proportional to square root of the number of trials) and reduce the surgical procedural times.

In any embodiment disclosed herein, various connections may be wired or wireless. Possible architectures include, but are not limited to:
In some embodiments, the data acquisition (DAQ) circuitry for the EMG leads/electrodes/signals may be located in the docking station. The EMG leads may connect directly to the docking station. In some embodiments, a hub may exist in a passive capacity, where it does not amplify or alter the EMG signal.

In some embodiments, the DAQ circuitry for the EMG may be located in the hub, and the hub receives and/or sends synchronization signal through a wireless connection with the docking station.

Some embodiments of the device may include optical capabilities. For example, the tip may include one or more small LEDs and photodiodes. Light can be applied to the tissue via the LED(s) and the reflected light measured by the photodiode(s). Different wavelengths of light may be used, including visible and/or nonvisible wavelengths. The reflectance of tissue at various wavelengths may be used to identify different tissues, including potentially tumor tissue, blood vessels, healthy tissue, ischemic tissue, necrotic tissue, cancerous tissue etc.

### Example of Data Processing System

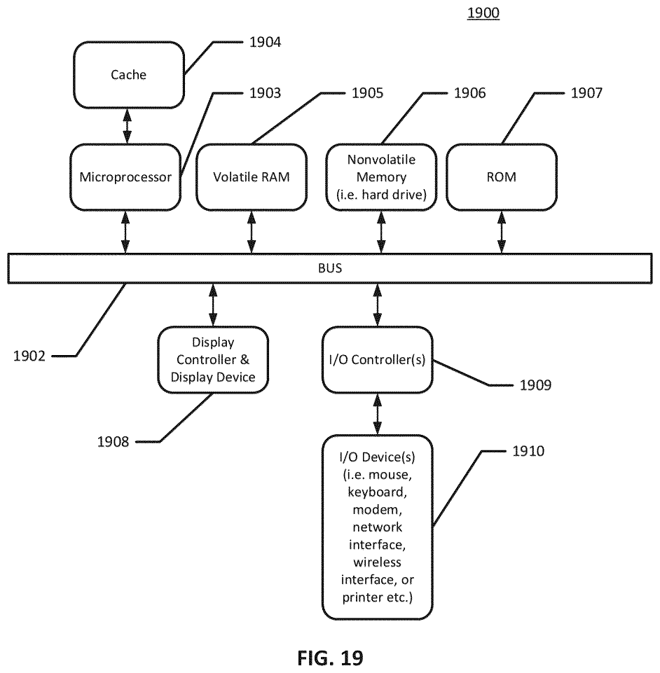

FIG. 19 is a block diagram of a data processing system, which may be used with any embodiment of the invention. For example, the system 2600 may be used as part of the controller or docking station. Note that while FIG. 26 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 19, the computer system 1900, which is a form of a data processing system, includes a bus or interconnect 1902 which is coupled to one or more microprocessors 1903 and a ROM 1907, a volatile RAM 1905, and a non-volatile memory 1906. The microprocessor 1903 is coupled to cache memory 1904. The bus 1902 interconnects these various components together and also interconnects these components 1903, 1907, 1905, and 1906 to a display controller and display device 1908, as well as to input/output (I/O) devices 1910, which may be mice, keyboards, modems, network interfaces, printers, and other devices which are well-known in the art.

Typically, the input/output devices 1910 are coupled to the system through input/output controllers 1909. The volatile RAM 1905 is typically implemented as dynamic RAM (DRAM) which requires power continuously in order to refresh or maintain the data in the memory. The non-volatile memory 1906 is typically a magnetic hard drive, a magnetic optical drive, an optical drive, solid-state flash memory or a DVD RAM or other type of memory system which maintains data even after power is removed from the system. Typically, the non-volatile memory will also be a random-access memory, although this is not required.

While FIG. 19 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1902 may include one or more buses connected to each other through various bridges, controllers, and/or adapters, as is well-known in the art. In one embodiment, the I/O controller 1909 includes a USB (Universal Serial Bus) adapter for controlling USB peripherals. Alternatively, I/O controller 1909 may include IEEE-1394 adapter, also known as Fire Wire adapter, for controlling FireWire devices, SPI (serial peripheral interface), I2C (inter-integrated circuit) or UART (universal asynchronous receiver/transmitter), or any other suitable technology. Wireless communication protocols may include Wi-Fi, Bluetooth, ZigBee, near-field, cellular and other protocols. The system may also include safety measures for Bluetooth (BT) or wireless communication failures between the hand-held stimulator and the docking station. Stacks, in both firmware and docking station software, provide a notification when there is a loss of pairing between the docking station and the stimulator. Additionally, the embodiment may include a 1 Hz “heartbeat”/watchdog communication over BT between the stimulator and docking station; if either of them stop functioning, this “heartbeat” will cease. In such cases, the hand-held stimulator will stop stimulation in progress and prevent any risk to the patient.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the Figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals-such as carrier waves, infrared signals, digital signals).

The processes or methods depicted in the preceding FIGS. may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

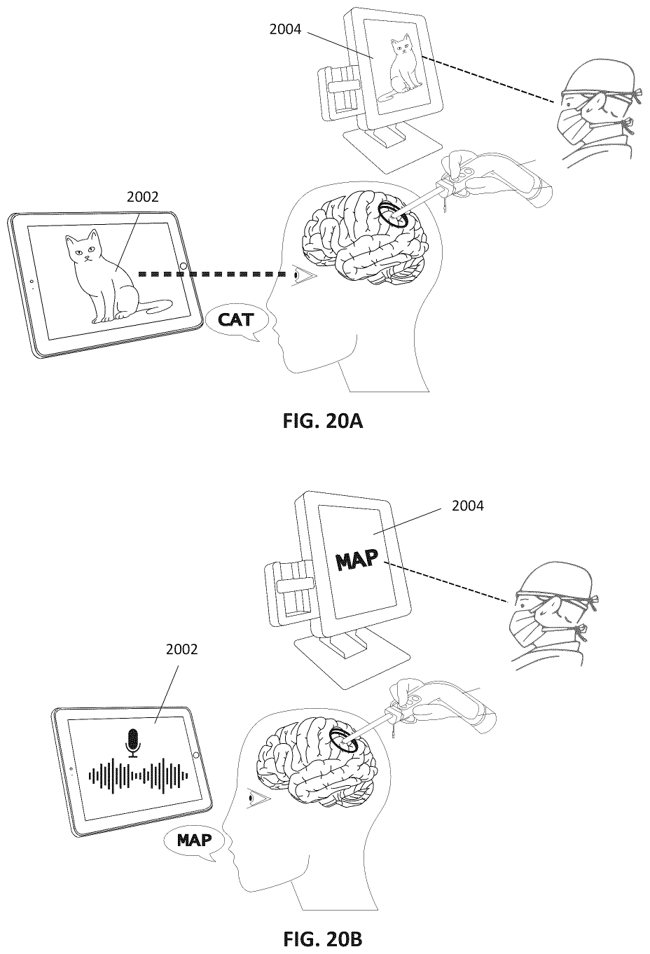

FIGS. 20A and 20B shows an embodiment of the device used in mapping the language processing, image processing and cognitive processing centers of the brain. In this embodiment, there may be an additional display or screen 2002 that is connected via a wired or wireless connection to the main docking station. The display may present preset patient stimuli (images, text, flashing lights, audio, etc.) that are synchronized with electrical stimulus output. The same stimulus may be presented on the docking station 2004 such that the surgeon is aware of what is being presented to the patient during stimulation and mapping during surgery.

In some embodiments, the preset patient stimuli (image, text, flashing lights, audio, etc.) can be user modifiable and customizable.

In some embodiments, the display may include a microphone that captures and records audio from the patient. The display may transmit this audio to another device such as a tablet, docking station or computer for processing.

The captured audio may be processed using natural language processing algorithms to determine various attributes of the audio spoken by the patient, such as whether the correct word was spoken, the cadence of the speech and other such parameters.

In some embodiments the display may include a camera that records the patient during the mapping procedure.

In some embodiments the recorded audio and video may be played back for review by the surgeon.

In some embodiments, the surgeon may manually enter the result of the electrical stimulation either via the touchscreen display on the docking station or via a voice command that is processed by the docking station. The recorded data may include the site of the stimulation (for example a marker with a number placed on the brain), the category of response from the patient due to electrical stimulation (for example: anomia, alexia, semantic or phonological paraphasia, etc.) and tally of the number of times a given site that is electrically stimulated produces a certain category of response.

In some embodiments, the additional display 2002 may be used pre-operatively in the same manner, where stimuli (image, text, flashing lights, audio, etc.) are presented to the patient to create a baseline for use during intra-operative mapping of the language processing, image processing and cognitive processing centers of the brain.

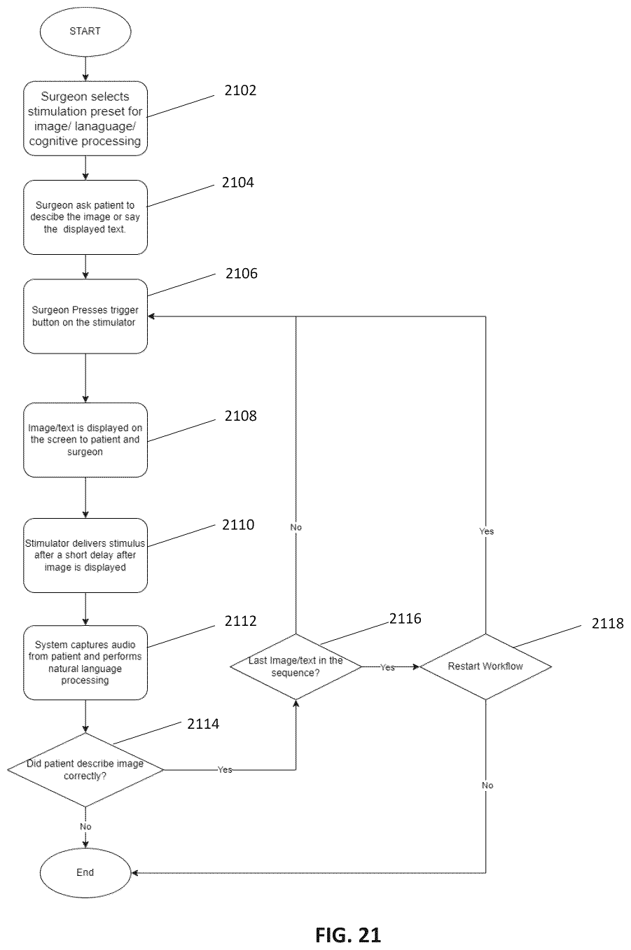

FIG. 21 shows the workflow for the mapping of the image, language and cognitive processing centers of the brain. The surgeon selects a preset for this mapping that configures the stimulus output pulse parameters as well the images and text that will be displayed on the screen and docking station (step 2102). The surgeon asks the patient to describe the image or repeat the text shown on the display (step 2104). The surgeon presses the trigger button on the stimulator which initiates the display of the image and/or text on the accessory display viewed by the patient and docking station viewed by the surgeon. (step 2106). The image/text is displayed to the patient and surgeon (step 2108). After a short delay the stimulator automatically delivers the stimulus current (step 2110). The system captures the audio from the patient and performs natural language processing (step 2112). The language processing algorithm determines if the audio from the patient matches certain criteria such as whether the words accurately describe the image or match the text displayed on the screen, cadence of speech, etc. (step 2114). The workflow will advance to display the next image and/or text in the sequence (step 2116). If it is the last item in the sequence then the surgeon has the option to restart the workflow or terminate it (step 2118).

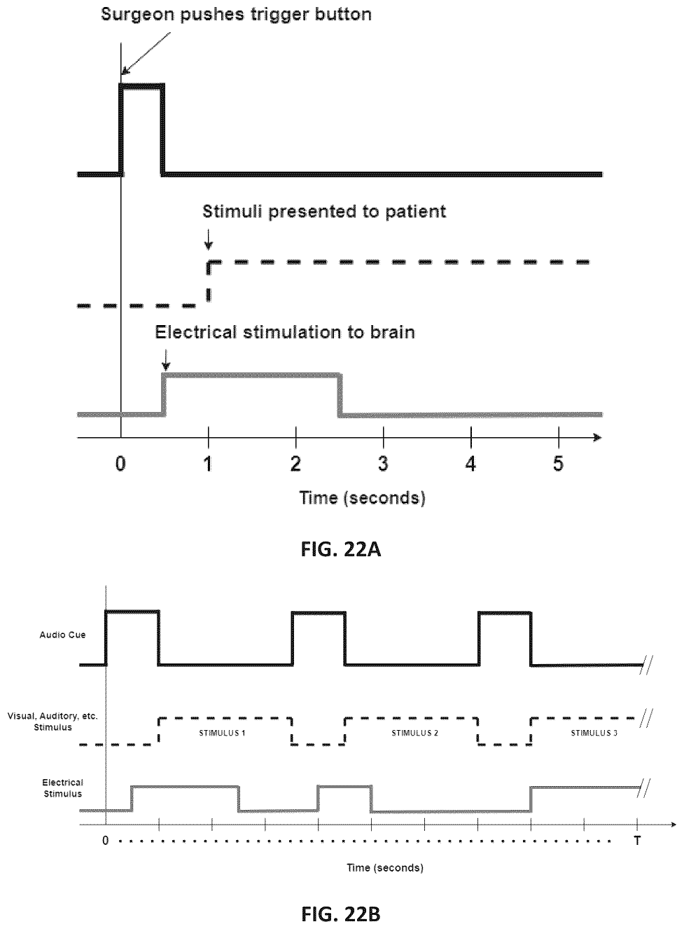

FIGS. 22A and 22B depict timing diagrams for possible implementations of the mapping scheme described in workflow shown in FIG. 21. In FIG. 22A, the delivery of the electrical stimulus to the brain tissue and the presentation of patient stimuli (images, cues, text, flashing lights, audio, etc.) are automatically triggered at configurable fixed delays from when the trigger button on the handheld stimulator is pressed. This allows for implementing various brain mapping schemes where the electrical stimulation of brain tissue using the hand-held stimulator is performed before, after or exactly at the same time that stimuli (images, cues, text, flashing lights, audio, etc.) are presented to the patient to optimize functional brain mapping and assist treatment.

FIG. 22B, the presentation of the patient stimulus would be synchronized with an audio cue. The surgeon manually delivers the electrical stimulus to the brain in a variable fashion with respect to the audio cue and presentation of patient stimulus. Furthermore, the duration of the stimulus may be variable as well and controllable by the surgeon via preset parameters on the docking station or via duration of contact of the electrical stimulator with tissue.

In other embodiments of the stimulation and brain mapping techniques may involve algorithms where signal averaging is used especially for the detection of evoked potential or evoked response for mapping of language and cognitive function, since the SNR is high and the signal is in the order of a few microvolts compared to millivolts for motor response. Time-linked averages may be used to measure neural responses and test the neural pathways that affect speech and cognitive function using EMG and ECOG scalp electrodes.

Similar to the detection of automated motor thresholds during cortical and subcortical mapping described in FIGS. 11-18, the stimulator device and docking station may be used to detect the evoked potentials for mapping the language, image processing and cognitive processing centers of the brain. These may be done by manual stimulation and visually looking for evoked functional response or may be automated. Both stimulation and observed biological response can be automated using machine learning and artificial intelligence algorithms. The automated stimulation and detection of evoked potentials is a significant advantage, because it enables neurosurgeons to make clinical decisions during surgery without the need of a neurologist or neurophysiologist (NP).

What is claimed is:

1. An apparatus for creating a closed-loop response feedback for an automated brain mapping system comprising of:
a neural stimulator;
a console docking station wirelessly communicating with the neural stimulator; and
one or more human response sensors configured to interface with the console docking station,
wherein said one or more human response sensors are triggered in response to an electrical signal delivered by said neural stimulator.

2. An apparatus as in claim 1 wherein said neural stimulator is a bipolar or monopolar.

3. An apparatus as in claim 1 wherein said human response sensors comprise of an electromyography (EMG) device to measure the electrical activity of muscles and nerves by stimulating regions of the motor cortex.

4. An apparatus as in claim 1 wherein said human response sensors comprise of a speech and language processing analyzer to interpret verbal reaction.

5. An apparatus as in claim 1 wherein said human response sensors comprise of an optical nerve measurement system to check reaction to stimulating regions of the visual cortex.

6. An apparatus as in claim 1 wherein said human response sensors comprise of an audiometer to check hearing by stimulating the auditory cortex.

7. An apparatus as in claim 1 wherein said human response sensors comprise of a neural stimulator is handheld.

8. An apparatus as in claim 1 wherein said human response sensors comprise of a neural stimulator is robotically operated.

9. A method of creating a closed-loop range finding feedback for an automated brain mapping system, comprising:
a neural stimulator
wirelessly communicating with
a console docking station
interface to human response sensors
interface to external sensors
wherein said modification instructions to the handheld neural stimulator are provided by information from either human response sensors, external sensors or combination thereof.

10. A method as in claim 9, wherein said modification instructions comprise of adjustment of the current setting.

11. A method as in claim 9, wherein said modification instructions comprise of adjusting the frequency.

12. A method as in claim 9, wherein said modification instructions comprise of repositioning the probe spatially.

13. A method as in claim 9, wherein said repositioning is on the cortical surface.

14. A method as in claim 9, wherein said repositioning is adjusting the subcortical depth of the stimulator.

15. A method as in claim 9, wherein said repositioning is on the cortical surface.

16. A method as in claim 9, wherein said neural stimulator is handheld.

17. A method as in claim 9, wherein the neural stimulator is robotically operated.

18. A method as in claim 17 wherein said robotically operated neural stimulators reposition the probe spatially based on the modification instructions.
